(19) United States
(12) Patent Application Publication
(10) Pub. No.: US 2020/0008676 A1
(43) Pub. Date: Jan. 9, 2020

(54) MEDICAL IMAGING APPARATUS COMPRISING PRIMARY MODULE AND SUPPLEMENTAL MODULE AND PROCESS THEREOF

(71) Applicant: Shanghai YUANWEI HEALTHCARE CONSULTING Inc., Shanghai (CN)

(72) Inventor: Yonghua Dong, Solon, OH (US)

(21) Appl. No.: 16/483,440

(22) PCT Filed: Feb. 28, 2018
(86) PCT No.: PCT/US18/20083

§ 371 Date: Aug. 4, 2019

Related U.S. Application Data
(60) Provisional application No. 62/464,968, filed on Feb. 28, 2017.

(51) Int. Cl. A61B 5/00; A61B 6/00; A61B 5/11

(52) U.S. Cl. CPC A61B 5/0035; A61B 5/0053; A61B 6/5229; A61B 6/505; A61B 6/463; A61B 5/055; A61B 5/0073; A61B 5/1128; A61B 5/0077; A61B 5/6889; A61B 5/4887; A61B 5/0095

(57) ABSTRACT

The present invention provides medical imaging apparatus comprising a primary module for imaging a patient and a supplemental module. A controlling circuitry activates the supplemental module shortly before, during, or shortly after an imaging process using the primary module. Information, data or images of the patient acquired with the supplemental module is registered to the primary image. The invention can aid a radiologist to avoid confusion, mismatching, and other errors when he is reading the primary image; and can help a doctor to establish a correlation between conditions revealed by the primary image, and cutaneous conditions and anatomical change revealed by the supplemental module.

### CROSS-REFERENCE TO RELATED APPLICATIONS

This application expressly claims all Paris Convention and related priority from U.S. Provisional Application for U.S. Patent Ser. No. 62/464,968 titled “Medical Imaging Apparatus and Method of Using the Same” and filed Feb. 28, 2017, which is incorporated by references as if set forth in its entirety.

### FIELD OF THE INVENTION

The present invention generally relates to a medical imaging apparatus comprising a primary module and a supplemental module and process thereof. Although the invention will be illustrated, explained and exemplified by a X-ray projection radiographing device, it should, be appreciated that the present invention can also be applied to other medical imaging apparatuses such as an ultrasound scanner, a X-ray CT scanner, a positron emission tomography (PET) device, a single-photon emission computed tomography (SPECT) device, a magnetic resonance imaging (MRI) scanner, an elastographer, a tactile imaging device, a photoacoustic imaging device, a thermoacoustic imaging device, a thermographing device, and the like.

### BACKGROUND OF THE INVENTION

Medical imaging can create visual representations of the interior of a body and physiological function of some organs or tissues for clinical analysis and medical intervention. In the clinical context, radiology or “clinical imaging” generally refers to “invisible light” medical imaging, and a radiologist is responsible for acquiring and/or interpreting the images, and sometimes even performing radiological interventions. In contrast, “visible light” medical imaging involves digital video or still pictures that can be seen without special equipment.

There exist some problems in radiology or “clinical imaging” of human internal structure. For example, human anatomy such as hand, feet, ear, eye, knee, eyeball and elbow etc have spatial features such as symmetry, chirality, alignment, and orientation. This could cause confusion, mismatching, and other errors when a radiologist is reading a clinical image. For example, left hand may be confused as right hand in X-ray image. FIG. 1 shows an X-ray image of a patient's hand. However, it is difficult to tell the X-ray image was taken from palmar aspect of the patient's left hand, or it was taken from dorsal aspect of the patient's right hand.

Advantageously, the present invention provides a medical imaging apparatus and a medical imaging process that can solve the aforementioned problems. In an exemplary embodiment of the present invention as shown in FIG. 2, a photo of a human hand with correct position and orientation is taken when X-ray imaging of the hand is being taken, and the photo is then registered to (and preferably integrated with) the corresponding X-ray image When a radiologist is reading the two side-by-side images (one visible and another X-ray) of the same hand in FIG. 2, he or she can readily tell the photo shows the palmar aspect of the patient's left hand, rather than the dorsal aspect of the patient's right hand. Therefore, the radiologist can ascertain that the X-ray image is an image of the left hand. The palm (volar) is the central region of the anterior part of the hand, and is located superficially to the metacarpus. A radiologist or even a layperson can appreciate that the skin in this area contains dermal papillae to increase friction. The opisthenar area (dorsal) is the corresponding area on the posterior part of the hand.

### SUMMARY OF THE INVENTION

One aspect of the present invention provides A medical imaging apparatus comprising a primary module for imaging a patient, wherein an image obtained by the primary module is defined as a primary image; a supplemental module that is different from the primary module, wherein the supplemental module acquires information, data or images of the patient that are associated with or supplemental to the primary image; a controlling circuitry configured to activate (or turn on) the supplemental module shortly before, during, or shortly after an imaging process using the primary module; and a processing circuitry that registers the information, data or images acquired from the supplemental module with the primary image.

Another aspect of the invention provides a medical imaging process using the above medical imaging apparatus and comprising steps of: (i) imaging a patient with a primary module to obtain a primary image, (ii) acquiring information, data or images of the patient that are associated with or supplemental to the primary image using a supplemental module that is different from the primary module shortly before, during, or shortly after the imaging in step (i); and (iii) registering the information, data or images acquired from the supplemental module with the primary image.

The above features and advantages and other features and advantages of the present invention are readily apparent from the following detailed description of the best modes for carrying out the invention when taken in connection with the accompanying drawings

### BRIEF DESCRIPTION OF THE SEVERAL VIEWS OF THE DRAWINGS

The present invention is illustrated by way of example, and not by way of limitation, in the figures of the accompanying drawings and in which like reference numerals refer to similar elements. All the figures are schematic and generally only show parts which are necessary in order to elucidate the invention. For simplicity and clarity of illustration, elements shown in the figures and discussed below have not necessarily been drawn to scale. Well-known structures and devices are shown in simplified form, omitted, or merely suggested, in order to avoid unnecessarily obscuring the present invention.

FIG. 1 shows an X-ray image of a patient's hand in the prior art.

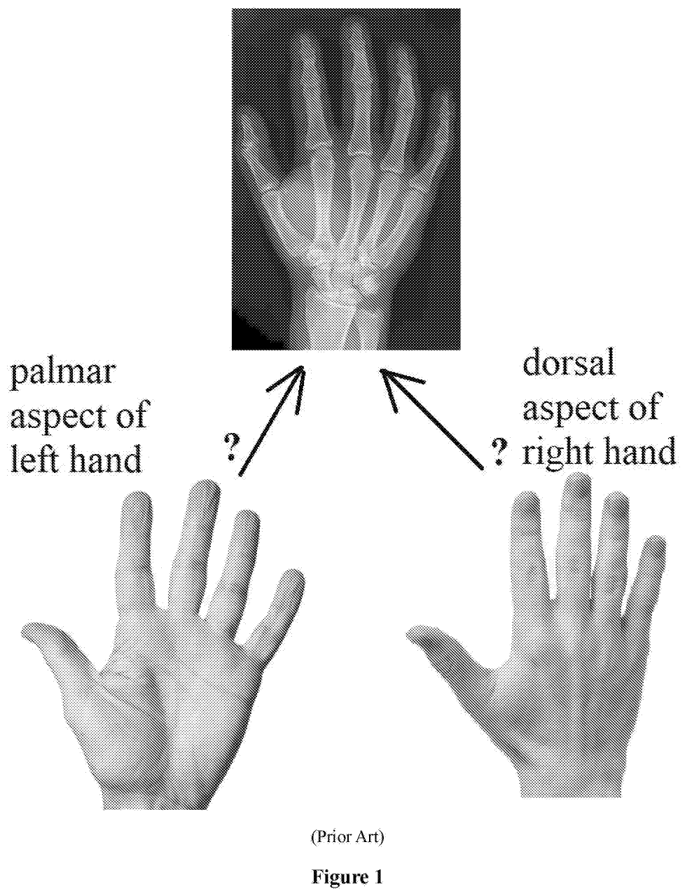

FIG. 2 is shows a photo of a human hand with correct position and orientation registered to its corresponding X-ray image in accordance with an exemplary embodiment of the present invention.

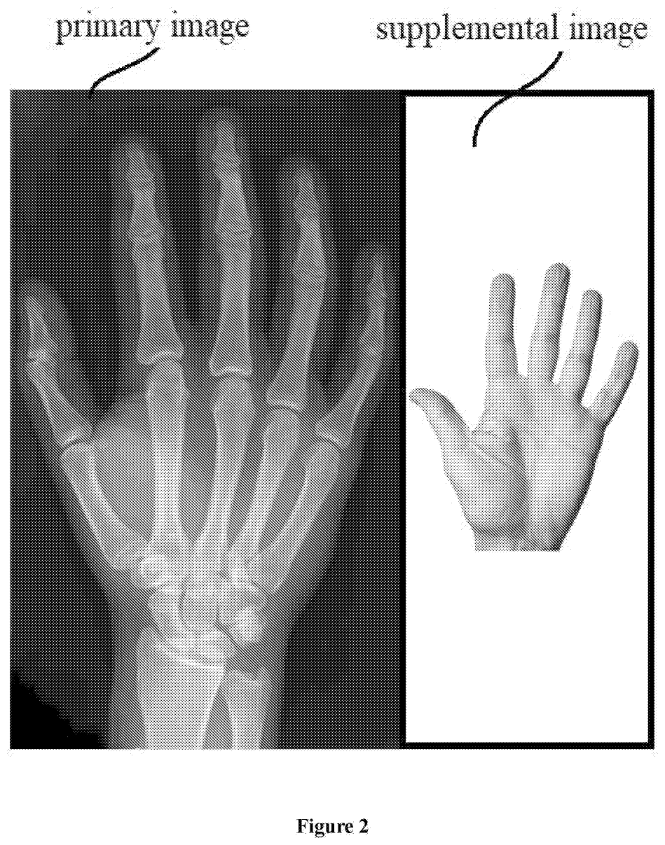

FIG. 3 schematically illustrates a medical imaging apparatus in accordance with an exemplary embodiment of the present invention.

FIG. 4 schematically an X-ray projection radiographing device as a primary module and a camera as the supplemental module in accordance with an exemplary embodiment of the present invention.

FIG. 5 depicts the acquirement of X-ray image and visible photo of a patient's left hand, and a photo of his face in accordance with an exemplary embodiment of the present invention.

FIG. 6 schematically shows other non-camera devices used as the supplemental module in accordance with an exemplary embodiment of the present invention.

FIG. 7 schematically illustrates various primary modules and the placement of the supplemental module in accordance with an exemplary embodiment of the present invention.

FIG. 8 schematically illustrates the acquirement of the thermographic image and visible photo of a patient's right breast, and a photo of her face in accordance with an exemplary embodiment of the present invention.

FIG. 9 illustrates an example of establishing a conelation between the conditions revealed by a primary image and a cutaneous symptom in accordance with an exemplary embodiment of the present invention.

### DETAILED DESCRIPTION OF THE PREFERRED EMBODIMENT

In the following description, for the purposes of explanation, numerous specific details are set forth in order to provide a thorough understanding of the present invention. It is apparent, however, to one skilled in the art that the present invention may be practiced without these specific details or with an equivalent arrangement.

Where a numerical range is disclosed herein, unless otherwise specified, such range is continuous, inclusive of both the minimum and maximum values of the range as well as every value between such minimum and maximum values. Still further, where a range refers to integers, only the integers from the minimum value to and including the maximum value of such range are included. In addition, where multiple ranges are provided to describe a feature or characteristic, such ranges can be combined.

It is also to be understood that the terminology used herein is for the purpose of describing particular embodiments only, and is not intended to limit the scope of the invention. For example, when an element is referred to as being “on”, “connected to”, or “coupled to” another element, it can be directly on, connected or coupled to the other element or intervening elements may be present. In contrast, when an element is referred to as being “directly on”, “directly connected to”, or “directly coupled to” another element, there are no intervening elements present.

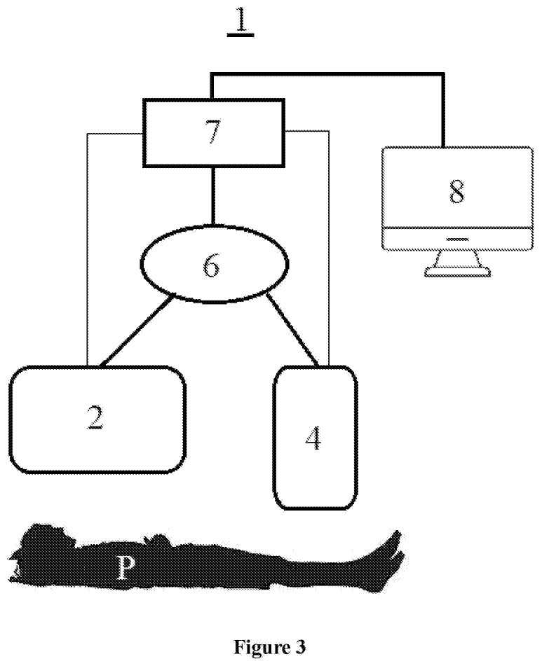

FIG. 3 schematically illustrates a medical imaging apparatus 1 of the invention. A primary module 2 is used for imaging a patient P, and the image obtained by the primary module 2 is defined as a primary image. A supplemental module 4 is different from the primary module 2, and the supplemental module 2 acquires information, data or images of the same patient that are associated with, or supplemental to, the primary image, and that may be referred to as supplemental information, supplemental data, and supplemental image. A controlling circuitry 6 is configured to activate (turn on) the supplemental module 4 shortly before, during, or shortly after an imaging process performed by the primary module 2. A processing circuitry or a processor 7 registers the information, data or images acquired from the supplemental module 4 with the primary image. A display 8 can display both the primary image and the information, data or images that are registered with the primary image for a radiologist to review. The phrase “shortly before the imaging process” is intended to mean less than 30 minutes, less than 20 minutes, less than 10 minutes, less than 5 minutes, than 3 minutes, less than 2 minutes, or less than 1 minute before the initiation of the imaging process performed by the primary module 2. The phrase “shortly after the imaging process” is intended to mean less than 30 minutes, less than 20 minutes, less than 10 minutes, less than 5 minutes, than 3 minutes, less than 2 minutes, or less than 1 minute after the completion of the imaging process performed by the primary module 2.

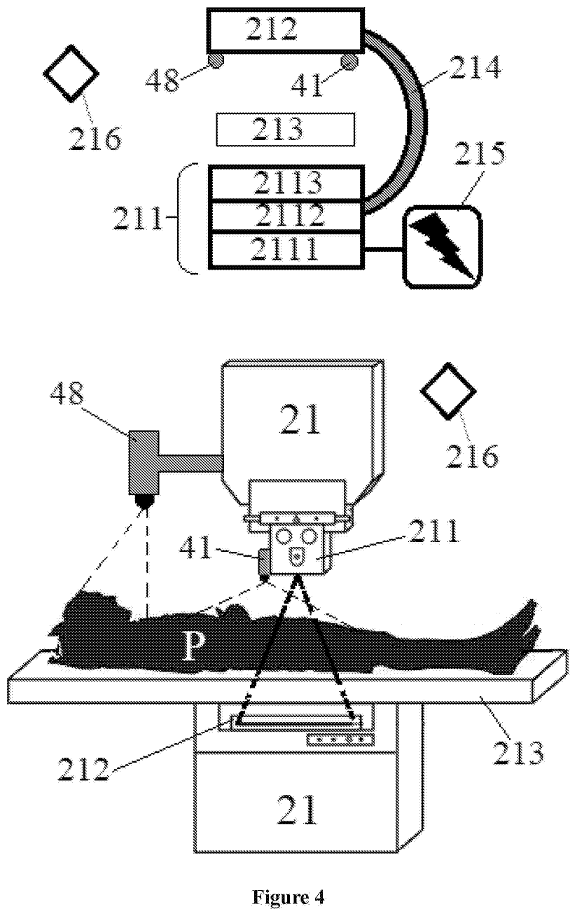

As shown in FIG. 4, an example of the primary module 2 is a known X-ray projection radiographing device 21, and the supplemental module 4 may include a medical camera 41 and an optional identity-recording camera 48 for recording a distinctive feature of the patient such as his/her face Cameras 41 and 48 are regular cameras with adjustable focusing capability, and function as the doctor's naked eyes. Cameras 41 and 48 are not anyone of the following: optical microscope, fluorescence microscope, electron'microscope or scanning probe microscope (SPM), magnifying glass, sheet magnifier, hand lens, reading stone, eyeglasses or spectacles, screen magnifier, and graphoscope. In FIG. 4, well-known structures and component in a typical X-ray imaging apparatus and a typical digital camera are shown in simplified form, omitted, or merely suggested, in order to avoid unnecessarily obscuring the most important aspects of the present invention. The device 21 uses electromagnetic radiation, specifically X-rays, to view the patient P's internal structure. Since human body is made up of various substances with differing densities, X-rays can be used to reveal the internal structure of the body by highlighting these differences of attenuation, based on that X-ray photons are absorbed more by denser substances (e.g. calcium-rich bones). In operation, a beam of X-rays is produced by an X-ray source 211 and is projected toward a part of the patient P. A certain amount of X-ray is absorbed by the patient P, which is dependent on the particular density and composition of that body part. The X-rays that pass through the body part are captured by an X-ray detector 212 (either photographic film or a digital detector, either flat or curved). The detector 212 can then provide a superimposed 2D representation of all the internal structures within that body part, thereby generating the primary image of the invention. If necessary, a radio contrast agent can be used to make the structures of interest stand out visually from their background.

The medical camera 41 may be a digital still camera, or a digital movie/video camera optionally synchronized with a sound recorder. The images and movies obtained from the medical camera 41 may demonstrate the following of an anatomical part (e.g. abdomen) of the patient P being examined by the primary module 2 such as X-ray device 21: (i) an apparent appearance and apparent shape, (ii) a position relative to the primary module 2 such as X-ray device 21, and/or (iii) an orientation relative to the primary module 2 such as X-ray device 21.

In some embodiments, cameras 41 and 48 may be combined or merged into one single camera to function as two. Medical camera 41 and identity-recording camera 48 may be attached to, or separated from, the X-ray device 21 When the cameras 41 and 48 are attached to X-ray device 21, one or two of them may be attached to X-ray source 211 and/or X-ray detector 212. When the cameras 41 and 48 are separated from X-ray device 21, they may have one or more separate stands or posts, or they can be mounted on a wall in the room, if desired The camera can even be handheld cameras with wired or wireless connection to the medical imaging system. The medical imaging apparatus a shown in FIG. 4 comprises processing circuitry configured to acquire the primary X-ray image from X-ray device 21 based on X-rays radiated onto patient P, and to acquire the supplemental image(s) from cameras 41 and 48.

Medical camera 41 and identity-recording camera 48 capture signals in the visible spectrum (visible to the human eye) Electromagnetic radiation in this spectrum has wavelengths from about 390 to 700 nm (e.g. 500-680 nm) and frequency in the vicinity of 430-770 THz. Cameras 41 and 48 can capture all the colors that the human eyes and brain can distinguish, including not only pure colors or spectral colors, but also unsaturated colors such as pink, or purple variations such as magenta, although they may be absent from the spectrum, because they can be made only by a mix of multiple wavelengths Cameras 41 and 48 can capture light onto photographic plate, photographic film, and/or electronic image sensor such as a charge coupled device (CCD) or a CMOS sensor

Other parts in X-ray device 21 include a tabletop 213, a high-voltage generator 215, and an injector 216. The tabletop 213 is a bed on which patient P is placed. In some embodiments, X-ray device 21 may include a C-arm 214 that holds X-ray source 211 and X-ray detector 212 and positions them in such a manner that they oppose each other while the patient P is interposed therebetween. Known mechanisms can be employed to rotate and move the C-arm 214, and to move the tabletop 213. The injector 216 is a device used for injecting a contrast agent through a catheter inserted in patient P. The injection of the contrast agent from the injector 216 may be executed according to an injection instruction received from a controlling circuitry. Alternatively, the injector 216 may start and stop the injection according to injection instructions directly input to the injector 216 from a radiologist.

X-ray source 211 includes an X-ray tube 2111, a collimator 2112, and a filter 2113. The high-voltage generator 215 is configured to generate a high voltage and to supply the generated high voltage to the X-ray tube 2111. The X-ray tube 2111 is configured to generate an X-ray by using the high voltage supplied thereto from the high-voltage generator 215. The collimator 2112 is configured to limit the X-rays generated by the X-ray tube 2111 so that the X-rays are selectively radiated onto a region from which an X-ray image is to be acquired. For example, the collimator 2112 includes four slidable limiting blades. Collimator 2112 limits the X-rays generated by the X-ray tube 2111 by sliding the limiting blades so that the X-rays are radiated onto the patient P. The limiting blades are each a plate-like member configured by using lead or the like and are provided near an X-ray radiation opening of the X-ray tube 2111, so as to be able to regulate the radiation range of the X-rays.

The filter 2113 is an X-ray filter used for adjusting the X-rays emitted from the X-ray tube 2111. For example, for the purpose of reducing the radiation exposure amount of the patient P and improving the image quality of image data, the filter 2113 is configured to reduce soft-ray components that are easily absorbed by patient P and to reduce high-energy component, that may degrade the contrast of X-ray images, by changing the characteristics of the X-rays passing through with the material and/or the thickness thereof. Further, the filter 2113 may be configured to attenuate the X-rays radiated from the X-ray tube 2111 onto patient P so as to have a predetermined distribution, by changing the radiation dose and the radiation range of the X-rays with the material, the thickness, and/or the position thereof.

The X-ray detector 212 is configured to detect X-rays that have passed through patient P. For example, the X-ray detector 212 includes detecting elements arranged in a matrix formation. The detecting elements are configured to convert the X-rays that have passed through the patient P into electric signals, to accumulate the electric signals, and to transmit the accumulated electric signals to an image data generating circuitry. For example, the image data generating circuitry generates the primary image by applying a current/voltage conversion, an Analog/Digital (A/D) conversion, and/or a parallel/serial conversion to the electric signals received from the X-ray detector 212. A storage circuitry may be employed to receive and store therein the image data generated by the image data generating circuitry, as well as the image data generated by cameras 41 and 48. An image processing circuitry may be employed to perform various types of image processing processes on the image data stored in the storage circuitry, for example, registering the information, data or images acquired from the supplemental module (e.g. the image data generated by cameras 41 and 48) with the primary image (e.g. the image data generated by X-ray device 21).

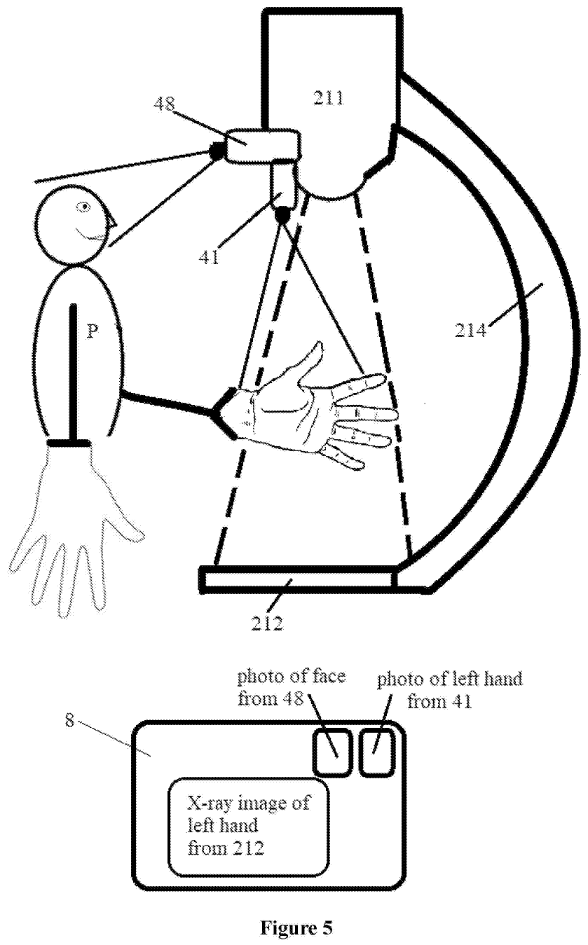

Referring now to FIG. 5, when a patient P is taking an. X-ray image of his left hand between X-ray source 211 and X-ray detector 212, an optional identity-recording camera 48 may take a photo of his face, and medical camera 41 may take a photo of his left hand. As a result, display 8 can display not only the primary image (i.e. X-ray image of his left hand) and the information, data or images (i.e. photo of his left hand from camera 41) that are registered with the primary image, but also an ID photo (i.e. photo of P's face from camera 48), for a radiologist to review. It should be appreciated that some supplemental images of the patient may help the physician to accurately determine the chirality and orientation of a body part in the primary image. An object or a system is chiral if it is distinguishable from its mirror image; that is, it cannot be superposed onto it. In human anatomy, hand, feet, ear, eye, knee, eyeball, kidney and elbow etc, have spatial features such as symmetry, chirality, alignment, and orientation.

The ID photo (i.e. photo of P's face from camera 48) may be more extensively used in a hospital environment, e.g. for the purpose of searching, identification verification, and error proofing in a hospital management system At the beginning of (or before) the primary imaging process, a photo of a patient's face is taken. Using image analysis and comparison technology, the face photo is then compared to a saved face photo of the same patient in the medical record. RIS/HIS/PACS of the hospital to verify the patient's identity. The medical record in RIS/HIS/PACS of the hospital with the same face may also be retrieved and reviewed by the radiologist, making sure that the primary imaging process to be completed is correct. After the examination is completed, the system may automatically compare the facial image acquired immediately before the primary imaging or a new photo, and the facial image that was previously saved in the system. When the patient's identification is confirmed, the system will allow the primary image to be incorporated in the patient's medical record. The present invention may be used for the purpose of identification verification and error proofing before a surgery starts. The system will acquire the patient's facial information and surgery site information before the surgery. The system will automatically search the patient's medical conditions, surgery site (particularly left side or right side of the patient's body), and whether the planned surgical procedure is correct or not.

As shown in FIG. 5, the primary image and the supplemental images are always bundled, associated and lined together, and displayed together on display 8 as well. With the aid of the side-by-side supplemental images, a radiologist can avoid confusion, mismatching and other errors when he/she is reading the primary image. Specifically, the radiologist will know who the patient is, and which of his hand (left or right) is shown with the X-ray image.

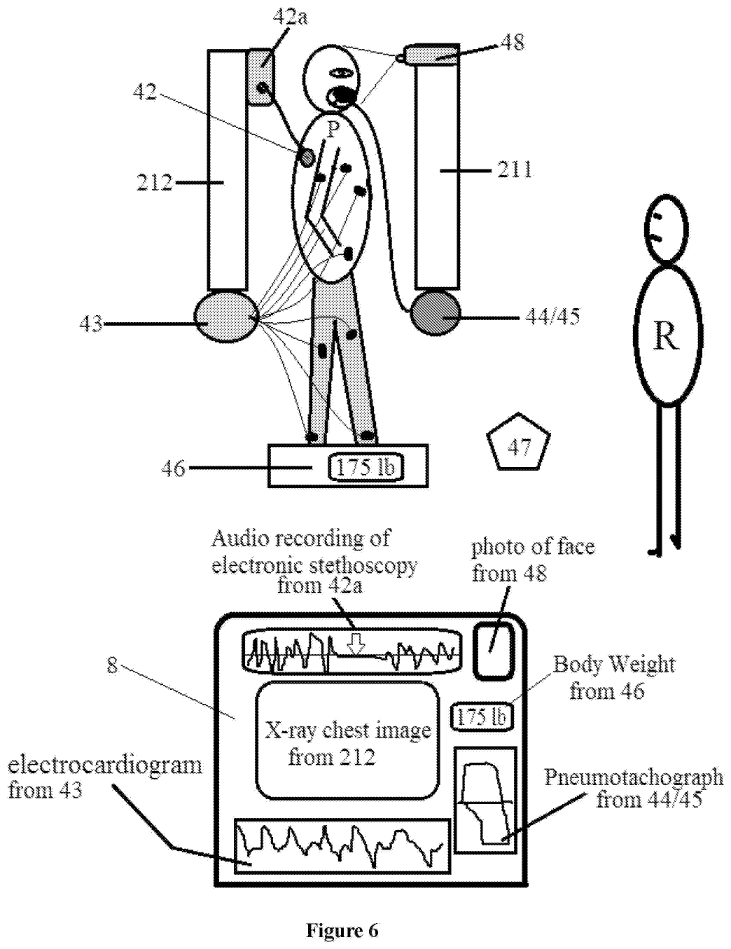

Referring to FIG. 6, the supplemental module 4 may include other devices such as a stethoscope 42 with a digital recording device 42a, an electrocardiography (ECG or EKG) device 43, a pulmonary function testing (PFT) device 44 such as a spirometer 45, a weighing scale 46, a sound recorder 47 for recording conversation between patient P and radiologist R, or any combination thereof. The stethoscope 42 is an acoustic device for auscultation, which is used by a physician to listen to the internal sounds (particularly lung and heart sounds) of an animal or human body. The sound may then be recorded by device 42a attached to 211/212. Intestines and blood flow in arteries and veins can also produce acoustic signals. A small disc-shaped resonator in 42 may be placed against the chest of the patient to measure the sound. If a diaphragm is placed on the patient, body sounds vibrate the diaphragm, creating acoustic pressure waves. If ra bell is placed on the patient, the vibrations of the skin directly produce acoustic pressure waves. The bell transmits low frequency sounds, while the diaphragm transmits higher frequency sounds. In an electronic stethoscope (or stethophone) 42, the sound levels is electronically amplified. Electronic stethoscopes 42 can also be used with Computer-aided Auscultation programs to analyze the recorded heart sounds pathological or innocent heart murmurs. The electronic stethoscope 42 may have direct audio output that can be used with an external recording device 42a. Controlling circuitry 6 may be configured to control the timing of when to trigger the operation of the primary module for imaging the chest. As shown on display 8, the audio recording of the chest sound may indicate (as a straight line segment) a duration when the patient was holding his or her breath. As shown on display 8, an arrow in the audio file points toward the time point within the duration when the primary image (chest X-ray image) was taken.

Electrocardiography (ECG or EKG) device 43 in FIG. 6 is attached to 211/212 and can record the electrical activity of the heart over a period of time using electrodes placed on the skin. These electrodes detect the tiny electrical changes on the skin that arise from the heart muscle's electrophysiologic pattern of depolarizing during, each heartbeat. In a conventional 12-lead ECG, 10 electrodes are placed on the patient's limbs and on the surface of the chest. Electrode RA is placed on the right arm, avoiding thick muscle; LA in the same location where. RA was placed, but on the left arm; RL on the right leg—lateral calf muscle; LL in the same location where RE was placed, but on the left leg; V1 in the fourth intercostal space (between ribs 4 and 5) just to the right of the sternum (breastbone); V2 in the fourth intercostal space (between ribs 4 and 5) just to the left of the sternum; V3 between leads V2 and V4; V4 in the fifth intercostal space (between ribs 5 and 6) in the mid-clavicular line; V5 horizontally even with V4, in the left anterior axillary line, and V6 horizontally even with V4 and V5 in the midaxillary line. The overall magnitude of the heart's electrical potential is then measured from 12 different angles (“leads”) and is recorded over a period of time (usually 10 seconds). In this way, the overall magnitude and direction of the heart's electrical depolarization is captured at each moment throughout the cardiac cycle. The graph of voltage versus time produced by this noninvasive medical procedure is referred to as an electrocardiogram, as shown on display 8. During each heartbeat, a healthy heart has an orderly progression of depolarization that starts with pacemaker cells in the sinoatrial node, spreads out through the atrium, passes through the atrioventricular node down into the bundle of His and into the Purkinje fibers, spreading down and to the left throughout the ventricles. This orderly pattern of depolarization gives rise to the characteristic ECG tracing. An ECG conveys a large amount of information about the structure of the heart and the function of its electrical conduction system. Among other things, an ECG can be used to measure the rate and rhythm of heartbeats, the size and position of the heart chambers, the presence of any damage to the heart's muscle cells or conduction system, the effects of cardiac drugs, and the function of implanted pacemakers.

Pulmonary function testing (PFT) device 44 such as a spirometer 45 in FIG. 6 is attached to 211/212 and can measure lung function, specifically the amount (volume) and/or speed (flow) of air that can be inhaled and exhaled. Spirometry is helpful in assessing breathing patterns that identify conditions such as asthma, pulmonary fibrosis, cystic fibrosis, and COPD. As shown on display 8, pneumotachographs are charts that plot the volume and flow of air coming in and out of the lungs from one inhalation and one exhalation. The most common parameters measured in spirometry are Vital capacity (VC), Forced vital capacity (FVC), Forced expiratory volume (FEV) at timed intervals of 0.5, 1.0 (FEV1), 2.0, and 3.0 seconds, forced expiratory flow 25-75% (FEF 25-75) and maximal voluntary ventilation (MVV), also known as Maximum breathing, capacity, Peak expiratory flow (PEF), Tidal volume (TV), Total lung capacity (TLC), Diffusing capacity (DLCO), Maximum voluntary ventilation (MVV), and Static lung compliance (Cst), among others. Spirometry can also be part of a bronchial challenge test, used to determine bronchial hyper responsiveness to either rigorous exercise, inhalation of cold/dry air, or with a pharmaceutical agent such as methacholine or histamine. To assess the reversibility of a particular condition, a bronchodilator is administered before performing another round of tests for comparison. This reversibility test, or a post bronchodilator test (Post BD), is an important part in diagnosing asthma versus COPD Other complementary lung functions tests include plethysmography and nitrogen washout.

The system as shown in FIG. 6 is able to provide a one-stop resolution for a patient suffering chest pain. A quick diagnosis can be accomplished by completing the chest X-ray or chest CT and CCG or EKG at one location. For example, when patient P is taking an X-ray image of his chest between X-ray source 211 and X-ray detector 212, one or more devices selected from 42/42a, 43, 44/45, 46, and 47 can be activated to work shortly before, during, or shortly after the X-Ray imaging of P's chest. As a result, display 8 can display part or all of the information, data or images that are acquired with devices 42/42a, 43, 44/45, 46 and 47 and that are registered with the primary image (i.e. the chest image), for a radiologist R to review.

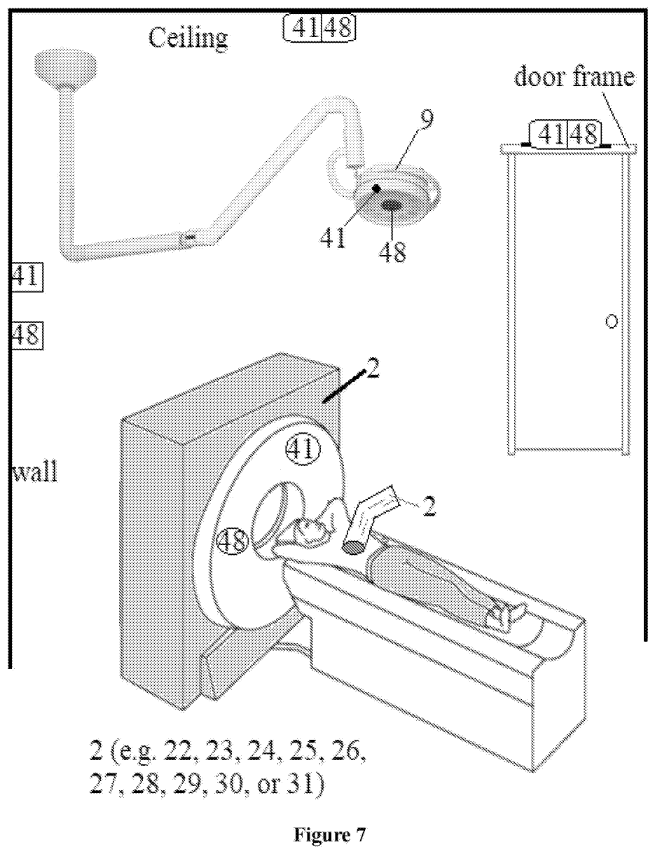

Referring to FIG. 7, in addition to X-ray projection radiographing device 21, the primary module 2 may be selected from an ultrasound scanner 22, a X-ray CT scanner 23, ra positron emission tomography (PET) device 24, a single-photon emission computed tomography (SPECT) device 25, a magnetic resonance imaging (MRI) scanner 26, a tactile imaging device 27, an elastographer 28, a photoacoustic imaging device 29, a thermoacoustic imaging device 30, a thermographing device 31, and the like, and any combination thereof.

Ultrasound scanner 22 generates sonography or ultrasonography to see internal structures of patient P such as tendons, muscles, joints, vessels and internal organs. Obstetric ultrasound is the practice of examining pregnant women using ultrasound. Ultrasonic images (sonograms) are made by sending pulses of ultrasound into tissue using a probe. The sound echoes off the tissue with different tissues reflecting varying degrees of sound. These echoes are recorded and displayed as an image to the operator. Many different types of images can be formed using sonographic instruments. The most well-known type is a B-mode image, which displays the acoustic impedance of a two-dimensional cross-section of tissue. Other types of image can display blood flow, motion of tissue over time, the location of blood, the presence of specific molecules, the stiffness of tissue, or the anatomy of a three-dimensional region

X-ray CT scanner 23 makes use of computer-processed combinations of many X-ray images taken from different angles to produce cross-sectional (tomographic) images (virtual “slices”) of specific areas of a scanned body part of patient P, allowing the radiologist to see internal structure of the body part without surgical cutting. Digital geometry processing is typically used to generate a three-dimensional image of the inside of the internal structure from a large series of two-dimensional radiographic images taken around a single axis of rotation.

Positron emission tomography (PET) device 24 is used to observe metabolic processes in the patient P's body. The PET system detects pairs of gamma rays emitted indirectly by a positron-emitting radionuclide (tracer), which is introduced into the body on a biologically active molecule. Three-dimensional images of tracer concentration within the body are then constructed by computer analysis. CT 23 and PET 24 can be combined into a PET-CT scanner, with which three dimensional PET imaging is accomplished with the aid of a CT X-ray scan performed on the patient during the same session using the same machine 2.

Single-photon emission computed tomography (SPECT) 25 is related to a 3D tomographic technique that uses gamma camera data from many projections and can be reconstructed in different planes. SPECT requires delivery of a gamma-emitting radioisotope or radionuclide into the patient P, normally through injection into the bloodstream. For example, a marker radioisotope is attached to a specific ligand to create a radioligand with properties enabling it to bind to certain types of tissues. Both ligand and radiopharmaceutical can be carried and bound to a place of interest in the patient's body, where the ligand concentration is seen by a gamma camera to generate the primary image of the invention. CT 23 and SPECT 25 can be combined into a SPECT-CT camera. For example, a dual detector head gamma camera combined with a CT scanner can be used for molecular imaging and providing localization of functional SPECT data.

Magnetic resonance imaging (MRI) machine 26 works based upon the science of nuclear magnetic resonance (NMR) Certain atomic nuclei can absorb and emit radio frequency energy when placed in an external magnetic field. For example, powerful magnets can polarize and excite hydrogen nuclei H⁺ (i.e., single protons) of water molecules in human tissue, producing a detectable signal which is spatially encoded, resulting in images of the body. The radio-frequency signal is received by antennas in close proximity to the anatomy in patient P being examined. Hydrogen atoms exist naturally in people and other biological organisms in abundance, particularly in water and fat. As such, most MRI scans essentially map the location of water and fat in the body. Pulses of radio waves excite the nuclear spin energy transition, and magnetic field gradients localize the signal in space. By varying the parameters of the pulse sequence, different contrasts can be generated between tissues based on the relaxation properties of the hydrogen atoms therein.

Tactile imaging device 27 translates the sense of touch into a digital image. The tactile image is a function of P (x,y,z), where P is the pressure on soft tissue surface under applied deformation and x,y,z are coordinates where pressure P was measured. Tactile imaging closely mimics manual palpation, since the probe of the device with a pressure sensor array mounted on its face acts similar to human fingers during clinical examination. The probe slightly deforms soft tissue and changes in the pressure pattern are measured. The device 27 is particularly useful for imaging of the prostate, breast, vagina and pelvic floor support structures, and myofascial trigger points in muscle.

Photoacoustic imaging device 29 works based on the photoacoustic effect. The device combines the advantages of optical absorption contrast with ultrasonic spatial resolution for deep imaging in (optical) diffusive or quasi-diffusive regime, Photoacoustic imaging can be used in vivo for tumor angiogenesis monitoring, blood oxygenation mapping, functional brain imaging, and skin melanoma detection, etc. In photoacoustic imaging, non-ionizing, laser pulses are delivered into biological tissues. In contrast, thermoacoustic imaging device 30 employs radio frequency pulses, and the technology is referred to as thermoacoustic imaging. Some of the delivered energy will be absorbed and converted into heat, leading to transient thermoelastic expansion and thus wideband (i.e. MHz) ultrasonic emission. The generated ultrasonic waves are detected by ultrasonic transducers and then analyzed to produce images Optical absorption is closely associated with physiological properties, such as hemoglobin concentration and oxygen saturation.

Other examples of the primary module 2 may include, but are not limited to, high frequency ultrasound (HIFU), intra-vascular ultrasound (IVUS), fluoroscopic imaging, isocentric fluoroscopy, bi-plane fluoroscopy, multi-slice computed tomography (MSCT), optical coherence tomography (OCT), diffuse optical tomography, electrical impedance tomography, optoacoustic imaging, ophthalmology, corneal topography, optical coherence tomography, and scanning laser ophthalmoscopy, among others.

Supplemental module 4 such as one or both of the medical camera 41 and identity-recording camera 48 may be mechanically attached to the primary module 2, a wall of a room where the primary module 2 is in, a door frame of a room where the primary module 2 is in, a ceiling of a room where the primary module 2 is in, a shadowless lamp or a surgical lighthead 9; or X-ray source, X-ray filter, and/or X-ray detector as described above. The cameras 41 and/or 48 can even be handheld cameras with wired or wireless connection to the medical imaging system of the present invention. For example, a medical professional such as a radiologist, a physician, a nurse, and a lab staff can use a handheld identity-recording camera 48 to a take a facial photo of patient P, shortly before, during, or shortly after an imaging process using the primary module 2.

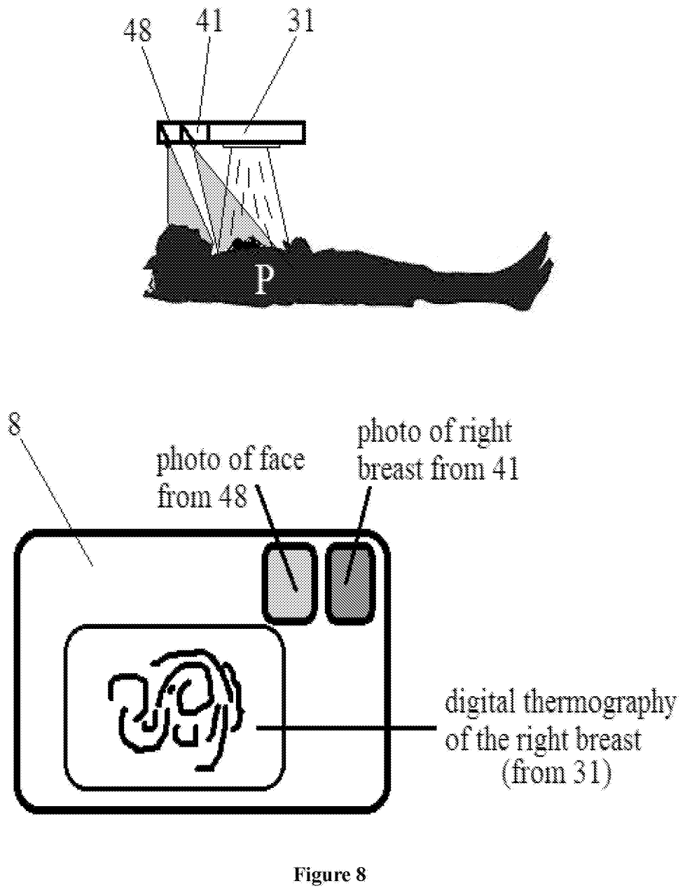

Taking thermographing device 31 as an example, and as illustrated in FIG. 8, device 31 is primarily used for breast imaging. There are three approaches: tele-thermography, contact thermography and dynamic angiothermography. FIG. 8 shows the approach of tele-thermography. These digital infrared imaging thermographic techniques are based on the principle that metabolic activity and vascular circulation in both pre-cancerous tissue and the area surrounding a developing breast cancer is almost always higher than in normal breast tissue. Cancerous tumors require an ever-increasing supply of nutrients and therefore increase circulation to their cells by holding open existing blood vessels, opening dormant vessels, and creating new ones (neo-angiogenesis theory). This process may result in an increase in regional surface temperatures of the breast. Some aspects of the dynamic angiothermography have advantages over the tele-thermography and contact thermography. First, the probes have better spatial resolution, contrastive performance, and the image is formed more quickly. Second, dynamic angiothermography is capable of identifying the thermal changes due to changes in vascular network to support the growth of the tumor/lesion. Instead of just recording the change in heat generated by the tumor, the image is able to identify changes due to the vascularization of the mammary gland. Referring to FIG. 8, when a patient P is taking a thermographic image of her right breast with digital thermographing device 31, an optional identity-recording camera 48 attached to device 31 may take a photo of her face, and medical camera 41 may take a photo of her right breast. As a result, display 8 can display not only the primary image (i.e. thermographic image of her right breast) and the information, data or images (i.e. photo of her right breast from camera 41) that are registered with the primary image, but also an ID photo (i.e. photo of P's face from camera 48), for a radiologist to review. As shown in FIG. 8, the primary image and the supplemental images are always bundled, associated and lined together, and displayed together as well on display 8. With the aid of the side-by-side supplemental images, a doctor can avoid confusion, mismatching and other errors when he/she is reading the primary image. Specifically, the doctor will know who the patient is, and which of her breast (left or right) is shown with the tele-thermography.

The present invention provides a medical imaging process using the medical imaging apparatus as described above. The process includes steps of: (i) imaging a patient with a primary module to obtain a primary image; (ii) acquiring information, data or images of the patient that are associated with or supplemental to the primary image using a supplemental module that is different from the primary module shortly before, during, or shortly after the imaging in step (i); and (iii) registering the information, data or images acquired from the supplemental module with the primary image. Optionally, the process may further comprise a step of displaying both the primary image and the information, data or images that are registered with the primary image on a same displaying device.

As described above, the primary module may include a X-ray projection radiographing device, an ultrasound scanner; a X-ray CT scanner, a positron emission tomography (PET) device, a single-photon emission computed tomography (SPECT) device, a magnetic resonance imaging (MRI) scanner, an elastographer, a tactile imaging device, a photoacoustic imaging device, a thermoacoustic imaging device, a thermographing device or any combination thereof. The supplemental module may include a medical camera, a stethoscope with a recording device, an electrocardiography (ECG or EKG) device, a pulmonary function testing (PFT) device such as a spirometer, a weighing scale, a sound recorder, or any combination thereof. For example, the medical camera may be a digital still camera, or a digital movie/video camera optionally synchronized with a sound recorder. The images and movies obtained from the medical camera can demonstrate: (i) an appearance and shape of an anatomical part of the patient being examined by the primary module, (ii) a position of an anatomical part of the patient being examined by the primary module, relative to the primary module, and/or (iii) an orientation of an anatomical part of the patient being examined by the primary module, relative to the primary module.

The aforementioned “anatomical part of the patient” may refer to head, face, ear, eye, cheek, nose, mouth, chin, neck, trunk, thorax, abdomen, pelvis, back, upper limb, pectoral girdle, axilla, arm, elbow, forearm, hand, lower limb, pelvic girdle, buttocks, hip, thigh, knee, leg, and foot etc.

The aforementioned “appearance and shape” may reveal one or more cutaneous conditions or symptoms (particularly colored symptoms) of the patient, as well as apparent anatomical change. As such, the medical imaging process of the invention may include an additional step of establishing a correlation between the conditions revealed by said primary image and said one or more cutaneous conditions or symptoms and said anatomical change. A cutaneous condition is any medical condition that affects the integumentary system that encloses the body and includes skin, hair, nails, and related muscle and glands. The major function of this system is as a barrier against the external environment. Possible diseases and injuries to the human integumentary system include Rash, Yeast, Athlete's foot, Infection, Sunburn, Skin cancer, Albinism, Acne, Herpes, Herpes labialis (cold sores), Impetigo, Rubella, Cancer, Psoriasis, Rabies, and Rosacea. Primary and secondary lesions are examples of cutaneous condition, and may include macule, patch, papule, plaque, nodule, vesicle, bulla, pustule, cyst, erosion, ulcer, fissure, weal, telangiectasia, burrow, scale, crust, lichenification, excoriation, induration, atrophy, maceration, umbilication, and phyma etc. The supplemental image of the invention can reveal lesion configuration such as agminate; annular or circinate; arciform or arcuate; digitate; discoid or nummular; figurate; guttate; gyrate; herpetiform, linear; mammillated, reticular or reticulated; serpiginous; stellate; targetoid; and verrucous. The supplemental image of the invention can also reveal lesion distribution such as symmetric; flexural; extensor; intertriginous; morbilliform; palmoplantar, periorificial, periungual/subungual; blaschkoid; photodistributed; zosteriform or dermatomal.

In some embodiments, the medical imaging process of the invention may include one or two of the following steps: recording a distinctive feature of the patient such as face with an identity-recording camera; and mechanically attaching one or both of the medical camera and identity-recording camera to the primary module, a wall of a room where the primary module is in, a door frame of a room where the primary module is in, a ceiling of a room where the primary module is in, a shadowless lamp or a surgical lighthead, X-ray source, X-ray filter, or X-ray detector.

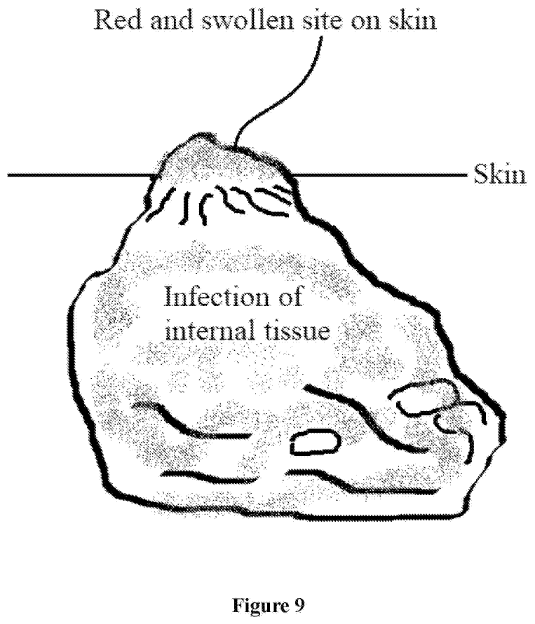

FIG. 9 illustrates an example of establishing a correlation between the conditions revealed by said primary image and one or more cutaneous conditions or symptoms. As shown in FIG. 9, infection of an internal tissue in an anatomical part of the patient may be revealed by MRI, and captured in a primary MRI image. The superficial appearance of the same anatomical part of the patient may have a sign of red and swollen skin, which will be captured by medical camera 41 in a supplemental image. A doctor can then establish a correlation between the internal infection symptom as revealed by the primary MRI image and the infection symptoms as revealed by the supplemental image or photo.

Techniques and technologies of the invention (particularly the embodiments as shown in FIGS. 3 rand 4) may be described herein in terms of functional and/or logical block components, and with reference to symbolic representations of operations, processing tasks, and functions that may be performed by various computing components or devices. Such operations, tasks, and functions are sometimes referred to as being computer-executed, computerized, processor-executed, software-implemented, or computer-implemented. It should be appreciated that the various block components shown in the figures may be realized by any number of hardware, software, and/or firmware components configured to perform the specified functions. For example, an embodiment of a system or a component may employ various integrated circuit components, e.g., memory elements, digital signal processing elements, logic elements, look-up tables, or the like, which may carry out a variety of functions under the control of one or more microprocessors or other control devices.

When implemented in software or firmware, various elements of the systems described herein are essentially the code segments or executable instructions that, when executed by one or more processor devices, cause the host computing system to perform the various tasks. In certain embodiments, the program or code segments are stored in a tangible processor-readable medium, which may include any medium that can store or transfer information. Examples of suitable forms of non-transitory and processor-readable media include an electronic circuit, a semiconductor memory device, a ROM, a flash memory, an erasable ROM (EROM), a floppy diskette, a CD-ROM, an optical disk, a hard disk, or the like.

In the foregoing specification, embodiments of the present invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicant to be the scope of the invention, is the literal and equivalent scope of the set of claims that issue from this application, in the specific form in which such claims issue, including any subsequent correction.

1. A medical imaging apparatus comprising
a primary module for imaging a patient, wherein an image obtained by the primary module is defined as a primary image;
a supplemental module that is different from the primary module, wherein the supplemental module acquires information, data or images of the patient that are associated with or supplemental to the primary image;
a controlling circuitry configured to activate the supplemental module shortly before, during, or shortly after an imaging process using the primary module; and
a processing circuitry that registers the information, data or images acquired from the supplemental module with the primary image.

2. The medical imaging apparatus according to claim 1, further comprising a display for displaying both'the primary image and the information, data or images that are registered with the primary image.

3. The medical imaging apparatus according to claim 1, wherein the primary module comprises a X-ray projection radiographing device, an ultrasound scanner, a X-ray CT scanner, a positron emission tomography (PET) device, a single-photon emission computed tomography (SPECT) device, a magnetic resonance imaging (MRI) scanner, an elastographer, a tactile imaging device, a photoacoustic imaging device, a thermoacoustic imaging device, a thermographing device or any combination thereof.

4. The medical imaging apparatus according to claim 1, wherein the supplemental module comprises a medical camera, a stethoscope with a recording device, an electrocardiography (ECG or EKG) device, a pulmonary function testing (PFT) device such as a spirometer, a weighing scale, a sound recorder, or any combination thereof.

5. The medical imaging apparatus according to claim 4, wherein the medical camera is a digital still camera, or a digital movie/video camera optionally synchronized with a sound recorder, and wherein images and movies obtained from the medical camera demonstrate
(i) an appearance and shape of an anatomical part of the patient being examined by the primary module,
(ii) a position of an anatomical part of the patient being examined by the primary module, relative to the primary module, and/or
(iii) an orientation of an anatomical part of the patient being examined by the primary module, relative to the primary module.

6. The medical imaging apparatus according to claim 4, further comprising an identity-recording camera for recording a distinctive feature of the patient such as face.

7. The medical imaging apparatus according to claim 6, wherein one or both of the medical camera and identity-recording camera are mechanically attached to the primary module, a wall of a room where the primary module is in, a door frame of a room where the primary module is in, a ceiling of a room where the primary module is in, a shadowless lamp or a surgical lighthead, X-ray source, X-ray filter, or X-ray detector.

8. The medical imaging apparatus according to claim 6, wherein the medical camera and the identity-recording camera are merged into one single camera.

9. A medical imaging process using the medical imaging apparatus of claim 1 comprising steps of:
(i) imaging a patient with a primary module to obtain a primary image;
(ii) acquiring information, data or images of the patient that are associated with or supplemental to the primary image using a supplemental module that is different from the primary module shortly before, during, or shortly after the imaging in step (i); and
(iii) registering the information, data or images acquired from the supplemental module with the primary image

10. The medical imaging process according to claim 9, further comprising a step of displaying both the primary image and the information, data or images that are registered with the primary image on a same displaying device.

11. The medical imaging process according to claim 9, wherein the primary module comprises a X-ray projection radiographing device, ran ultrasound scanner, a X-ray CT scanner, a positron emission tomography (PET) device, a single-photon emission computed tomography (SPECT) device, a magnetic resonance imaging (kap scanner, an elastographer, a tactile imaging device, a photoacoustic imaging device, a thermoacoustic imaging device, a thermographing device or any combination thereof.

12. The medical imaging process according to claim 9, wherein the supplemental module comprises a medical camera, a stethoscope with a recording device, an electrocardiography (ECG or EKG) device, a pulmonary function testing (PFT) device such as a spirometer, a weighing;scale, a sound recorder, or any combination thereof.

13. The medical imaging process according to claim 9, wherein the medical camera is a digital still camera, or a digital movie/video camera optionally synchronized with a sound recorder, and wherein images and movies obtained from the medical camera demonstrate
(i) an appearance and shape of an anatomical part of the patient being examined by the primary module,
(ii) a position of an anatomical part of the patient being examined by the primary module, relative to the primary module, and/or
(iii) an orientation of an anatomical part of the patient being examined by the primary module, relative to the primary module.

14. The medical imaging process according to claim 13, wherein said appearance reveals one or more cutaneous conditions of the patient, and said shape reveals an anatomical change, further comprising a step of
establishing a correlation between conditions revealed by said primary image and said one or more cutaneous conditions and said anatomical change.

15. The medical imaging process according to claim 14, wherein said one or more cutaneous conditions are Rash, Yeast, Athlete's foot, Infection, Sunburn, Skin cancer, Albinism, Acne, Herpes, Herpes labialis (cold sores), Impetigo, Rubella, Cancer, Psoriasis, Rabies, and Rosacea; lesions including macule, patch, papule, plaque, nodule, vesicle, bulla, pustule, cyst, erosion, ulcer, fissure, weal, telangiectasia, burrow, scale, crust, lichenification, excoriation, induration, atrophy, maceration, umbilication, and phyma; as well as lesion configurations and distributions.

16. The medical imaging process according to claim 13, further comprising a step of
recording a distinctive feature of the patient such as face with an identity-recording camera; and
optionally verifying the patient's identification by comparing said distinctive feature with a distinctive feature that was previously saved in a medical record.

17. The medical imaging process according to claim 16, further comprising a step of
mechanically attaching one or both of the medical camera and identity-recording camera to the primary module, a wall of a room where the primary module is in, a door frame of a room where the primary module is in, a ceiling of a room where the primary module is in, a shadowless lamp or a surgical lighthead, X-ray source, X-ray filter, or X-ray detector.

18. The medical imaging process according to claim 16, wherein the medical camera and the identity-recording camera are merged into one single camera.
